(19) United States
(12) Patent Application Publication
(10) Pub. No.: US 2010/0168053 A1
(43) Pub. Date: Jul. 1, 2010

(54) ORAL DELIVERY SYSTEM FOR METHYLCOBALAMIN TO TREAT DISORDERS

(75) Inventor: STAN KURTZ, Tarzana, CA (US)

Correspondence Address: FULBRIGHT AND JAWORSKI LLP, 555 S. FLOWER STREET, 41ST FLOOR, LOS ANGELES, CA, 90071, US

(73) Assignee: REVITAPOP, TARZANA, CA (US)

(21) Appl. No.: 12/723,421

(22) Filed: Mar. 12, 2010

Related U.S. Application Data
(63) Continuation-in-part of application No. 12/077,296, filed on Mar. 17, 2008.

(51) Int. Cl. A61K 31/7056; C07F 15/06; A61P 25/22; A61P 25/24; A61P 25/00; A61P 25/14; A61P 1/00; A61P 31/12; A61P 29/00; A61P 11/06; A61P 25/06; A61P 37/00; A61P 3/02; B29C 39/02

(52) U.S. Cl. 514/52; 548/108; 264/319

(57) ABSTRACT

A method of treating a disorder by lollipop administering methylcobalamin, with or without folinic acid by direct delivery to the trigeminal nerve. The disorders addressed are: a) attention deficit hyperactivity disorder (ADHD); b) anxiety, depression, stress and chronic stress; c) socialization problems, mood problems, behavior problems, memory problems; d) dyslexia, depth perception problems, color viewing problems, visual and auditory processing problems, light modulation problems, night vision problems; e) speech problems such as finding words, apraxia, and articulation problems, sleep regulation problems, eye or muscle movement problems; and f) chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, autoimmune problems. In some embodiments, the disorders that are particularly addressed are ADHD, anxiety, stress and chronic stress, and irritable bowel. A lollipop for treating a psychological or neuro-physiological disorder and method of making thereof.

### CROSS REFERENCE TO RELATED APPLICATIONS

The present invention is a Continuation-in-Part of U.S. application Ser. No. 12/077,296, filed Mar. 17, 2008, entitled, “USE OF METHYLCOBALAMIN NASAL SPRAY TO TREAT DISORDERS,” the contents of which are incorporated by reference in their entirety.

### FIELD OF INVENTION

This disclosure relates generally to oral administration via lollipop including methylcobalamin for treatment of psychological, neuro-physiological, and chronic disorders and diseases.

### BACKGROUND

Attention deficit hyperactivity disorder (“ADHD”), also called attention deficit disorder (“ADD”), is a neurobehavioral condition of children and adults that is characterized by a chronic level of inattention, hyperactivity, and impulsivity. Three types of ADHD are recognized. In the predominantly inattentive type, a person can have six or more of the following disruptive and age-inappropriate symptoms: difficulty paying attention to details, difficulty keeping attention on tasks, difficulty following instructions, difficulty organizing activities, difficulty following conversations, being easily distracted, and forgetful of daily routines. In the predominantly hyperactive-impulsive type, a person can have six or more of the following disruptive and age-inappropriate symptoms: fidgeting often, inappropriate running about, trouble playing or enjoying leisure activities quietly, excessive talking, blurting out answers, trouble waiting turn, and interrupting others. In the combined type, both inattentive and hyperactive-impulsive behaviors can be present. The Diagnostic & Statistical Manual for Mental Disorders (DSM-IV-TR), 2000, provides various criteria for diagnosing ADHD disorders.

Irritable bowel, or irritable bowel syndrome, is a disease characterized by abdominal cramping, bloating, flatulence, chronic constipation and/or diarrhea, and mucus in the stool.

Anxiety is a disorder characterized by persistent, irrational fear or worry. Examples of anxiety disorders include post-traumatic stress disorder and phobias such as irrational fear of spiders or open spaces. Chronic stress is an ongoing psychological and physiological state, also known as the “fight or flight” response, to various stimuli. Symptoms of chronic stress include cognitive, emotional, behavioral and physical problems.

Methylcobalamin is a vitamin B12 derivative that mediates methyl group transfer in the metabolic generation of methionine from homocysteine. Like vitamin B12, methylcobalamin is based on a corrin ring and has the formula C₆₃H₉₁CoN₁₃O₁₄P.

Autism is a developmental disorder characterized by impaired social interaction, repetitive or severely limited activities and interests, and verbal and nonverbal communication problems. Based on studies of oxidative stress and methionine synthase, subcutaneous injection of methylcobalamin has been found to be helpful in children with autism. Studies on the sublingual administration of cyanocobalamin, a different vitamin B12 derivative, have suggested that this route of administration is similar in efficacy to intravenous administration of cyanocobalamin, and that sublingual administration of hydroxycobalamin can provide a cobalamin normalizing effect that other routes of administration do not seem to have.

Methylcobalamin, not administered orally, has been reported to help improve oxidative status in children with autism, and is suggested to be an antiviral for AIDS, to be helpful in fighting cancer, peripheral neuropathy, and autonomic dysregulation, and to help improve DNA methylation and nerve regeneration. Oral administration via lollipop of methylcobalamin may be an effective method of treatment of these conditions as well as ADHD and other illnesses and disorders, as herein described.

Nasal administration of methylcobalamin may result in a portion of a sprayed dosage either being swallowed, so that the digestive tract destroys its effectiveness, or a portion may run from the nose, also reducing effectiveness. Methylcobalamin has a red coloration, which may give the undesirable impression of a bloody nose.

Lozenge administration of methylcobalamin may be used, but may be less reliable, given the tendency to chew tablets (especially with children) and pass a portion of the dose into the digestive tract, which may reduce efficacy.

Injected methylcobalamin, sometimes performed daily, may be an unpleasant experience, especially for children or adults with psychological or neuro-physiological disorders, such as, for example, autism spectrum disorders. There is a need, therefore, for a method of delivery of methylcobalamin that is acceptable to the user and provides a reliable dosage delivery.

### SUMMARY OF THE DISCLOSURE

In one aspect, a method of treating a person having a psychological or neuro-physiological disorder is provided. The method comprises orally administering methylcobalamin, or a pharmaceutically acceptable salt thereof, via lollipop to a person in need of such treatment in an amount sufficient to treat the disorder in the person. The disorder can be: a) ADHD; b) anxiety, depression, stress and chronic stress; c) socialization problems, mood problems, behavior problems, memory problems; d) dyslexia, depth perception problems, color viewing problems, visual and auditory processing problems, light modulation problems, night vision problems; e) speech problems such as finding words, apraxia, and articulation problems, sleep regulation problems, eye or muscle movement problems; and f) chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, autoimmune problems.

In certain embodiments, the disorders that are particularly addressed are ADHD, anxiety, stress and chronic stress, and irritable bowel.

In various embodiments, the treatment reduces one or more symptoms or characteristics of the particular disorder. For example, when the disorder is ADHD, the treatment can reduce hyperactivity symptoms of ADHD and/or inattentiveness symptoms of ADHD. The methylcobalamin, or pharmaceutically acceptable salt thereof, can be administered with or without folinic acid in various embodiments.

The novel features which are believed to be characteristic of the invention, both as to its composition and method of use, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 illustrates a method of making a lollipop for orally treating a psychological or neurophysiological disorder in accordance with the disclosure

FIG. 2 is a topograph showing a subject's theta wave/beta wave activity ratio before lollipop administration of methylcobalamin.

FIG. 3 is a topograph showing theta wave/beta wave activity ratio of the same subject as in FIG. 1 after lollipop administration of methylcobalamin.

### DETAILED DESCRIPTION

A method of treating certain psychological or neuro-physiological disorders is provided. In various embodiments, the method comprises orally administering, e.g., via lollipop, methylcobalamin or a pharmaceutically acceptable salt thereof, in an amount sufficient to treat one or more disorders observed in a person. As used herein, the term “disorder” broadly refers to a syndrome, condition, chronic illness or particular disorder. The term “treat” means to reduce one or more symptoms or characteristics of the disorder. For example, symptoms of hyperactivity or inattentiveness in ADHD can be reduced. By “reduce” is meant decreasing the number of events or the severity of a symptom or characteristic, or both. The term “therapeutically effective amount” means an amount sufficient to reduce one or more symptoms or characteristics of ADHD or other disorder.

The disorders can be any of the following: a) ADHD; b) anxiety, depression, stress and chronic stress; c) socialization problems, mood problems, behavior problems, memory problems; d) dyslexia, depth perception problems, color viewing problems, visual and auditory processing problems, light modulation problems, night vision problems; e) speech problems such as finding words, apraxia, and articulation problems, sleep regulation problems, eye or muscle movement problems; and f) chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, autoimmune problems. In some embodiments, the disorders that are particularly addressed are ADHD, anxiety, stress and chronic stress, and irritable bowel.

In addition to reducing symptoms or characteristics of the various disorders listed herein, lollipop administration of methylcobalamin may also assist in nerve growth, improve the ability to process sounds and modulate background noise, block the effects of alcohol, or reduce the desire for alcohol, coffee, marijuana, methamphetamines, and other addictive drugs and behaviors. Lollipop administration with reliable dosage control of methylcobalamin may also lead to improved brain synchronicity, improved brain blood flow, or reduced post-anesthetic recovery times and symptoms. Some additional symptoms that may be treated or reduced by methylcobalamin oral administration include white coated tongue, ocular stress (eye “twittering”), night vision challenges, food sensitivities, throat infections, trouble sleeping or waking up in the morning, and difficulty maintaining weight. Eye “twittering” is a condition in which a person has difficulty keeping his or her eyes fixed on one location, and instead frequently moves the eyes from place to place.

Lollipop administration of methylcobalamin may reduce a characteristic associated with a disorder even if there is no formal medical diagnosis of the disorder. For example, oral administration of methylcobalamin may reduce the pain associated with inflammation and autoimmune responses even though the level of inflammation or autoimmune response is less than the level required for a medical diagnosis. Similarly, oral administration of methylcobalamin may relieve, for example, anxiety, inattentiveness, hyperactivity, impulsivity, vision, and digestion in a subject even though these symptoms are not clearly associated with a medical disorder or diagnosis. Oral administration of methylcobalamin may also raise energy levels, which can provide benefits to a subject whether or not diagnosed with a disorder.

In a person responsive to methylcobalamin, the effects of methylcobalmin administered nasally may begin to occur within minutes of administration. For example, QEEG measurements of theta wave activity dependence on nasal methylcobalamin showed that methylcobalamin oral administration via lollipop (described below) works almost immediately. In various experiments, subjects reported feeling better in minutes and sometimes seconds. Thus, improvements can occur within the first hour of administration.

Although the effects of a typical administration can last about 24 hours, every person is different. Thus, some people can become depleted of available methylcobalamin in hours or less, while others can feel the benefits for days. The dosage and frequency of methylcobalamin administration can decrease over time, particularly with changing lifestyle or eating habits. For example, reducing intake of milk and wheat products, complex carbohydrates and starches, and improvements to intestinal flora may reduce the need for methylcobalamin.

It appears that some individuals do not respond to sublingual administration of methylcobalamin. For example, self-reporting by individuals has indicated that only a few people appear to significantly benefit from sublingual methylcobalamin administration. Anecdotal reports suggest that responders to methylcobalamin administered via lollipop do not respond to sublingual or nasal administration. A deficiency in certain types of bacteria of the intestinal flora and mucosa might have a possible role as well in reducing the effectiveness of sublingual or nasal administration.

There appear to be four types of responders to methylcobalamin based on observations of autistic persons: a) people who respond better to lollipop administration of methylcobalamin; b) people who respond better to a subcutaneous administration of methylcobalamin; c) people who respond to either mode of administering methylcobalamin; d) people who respond to neither mode of administering methylcobalamin. People with autism may respond better to methylcobalamin administered by lollipop.

In addition, it appears that lollipop or lozenge oral administration of methylcobalamin may be more effective than nasal administration of methylcobalamin. The rate of delivery of methylcobalamin is more gradual, and is delivered directly to the trigeminal nerves of the tongue, facilitating a direct pathway to the brain. The gradual administration via lollipop to the tongue may ensure that a greater fraction of the dose passes directly to the nervous system, whereas nasal spray absorption, for instance, is a short interval administration of dosage, of which a large fraction may be lost to the digestive tract, with less benefit of absorption.

Folinic acid, or a pharmaceutically acceptable salt thereof, can be added as a component of a lollipop containing methylcobalamin. Folinic acid can aid in the utilization of methylcobalamin, and may maintain methylcobalamin in the body for a greater period of time. If any hyperactivity or negative symptoms occur, the lollipop without folinic acid may be administered and evaluated.

Compositions for lollipop administration can, for example, be prepared by dissolving, dispersing, mixing or incorporating methylcobalamin (with or without folinic acid) and optional pharmaceutical adjuvants in a traditional candy recipe for a lollipop which may contain sugar or other sweeteners, emulsifiers, flavors, or dyes. In one representation, the lollipop is made with brown rice syrup, evaporated cane juice, fruit juice, sorbitol, or the like, citric acid and natural flavors. The methycobalamin (with or without folinic acid or other beneficial salts) is added to the lollipop mixture to thereby form a suspension of methycobalamin within a lollipop. If desired, the composition to be administered orally by lollipop can also contain minor amounts of nontoxic auxiliary substances such as wetting or emulsifying agents, pH buffering agents and the like, for example, sodium acetate, sorbitan mono-laurate, triethanolamine sodium acetate, triethanolamine oleate, potassium sorbate, glycerin, lecithin, etc. Actual methods of preparing such dosage forms are known, or will be apparent, to those skilled in this art; for example, see Remington's Pharmaceutical Sciences. Any adjuvant, excipient or auxiliary substance is contemplated so long as it does not prevent the effectiveness of lollipop administered methylcobalamin, and preferably, does not irritate the digestive tract.

The compositions disclosed herein may be formulated in neutral or salt form. Pharmaceutically-acceptable salts include acid addition salts that are formed with inorganic acids such as, for example, hydrochloric or phosphoric acids, or such organic acids as acetic, oxalic, tartaric, mandelic, and the like. Pharmaceutically-acceptable salts can also be derived from inorganic bases such as, for example, sodium, potassium, ammonium, calcium, or ferric hydroxides, and such organic bases as isopropylamine, trimethylamine, histidine, procaine and the like. The term “pharmaceutically-acceptable” refers to substances and compositions that do not produce an allergic or similar untoward reaction when administered to humans.

Methylcobalamin-containing lollipops, with or without folinic acid, can be obtained from various “autism friendly” and developmental disorders-focused compounding pharmacies. For example, one such pharmacy to compound methylcobalamin is Coastal Compounding Pharmacy (Savannah, Ga., USA) (on the World Wide Web at coastalcompounding.com).

Dosages of methycobalamin may vary in the range from about 0.1 mg to about 10 mg per lollipop.

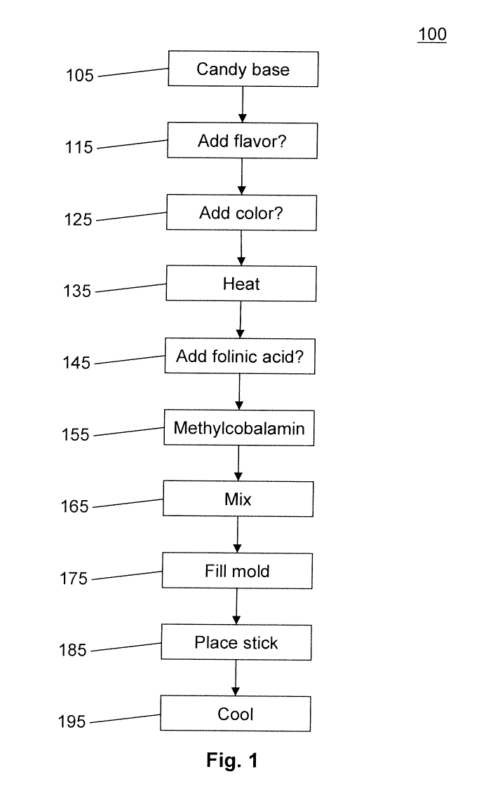

In a embodiment, FIG. 1 illustrates a method 100 of making a lollipop for orally treating a psychological or neurophysiological disorder. Method 100 includes providing a candy base (block 105), optionally adding a flavoring (block 115) to the candy base, optionally adding a dye coloring (block 125) to the candy base and heating to form a liquid (block 135). The candy base may be, for example, at least one of an evaporated cane juice, a brown rice syrup, sorbitol and an emulsified sugar. Folinic acid and/or a pharmaceutically acceptable salt of folinic acid may be optionally added (block 145) into the liquid in a selected amount to provide a desired concentration and dosage in the finished lollipop. Methylcobalamin and/or a pharmaceutically acceptable salt of methylcobalamin may be added (block 155) and into the liquid in a selected amount to provide a desired concentration and dosage in the finished lollipop. The liquefied composition may be mixed (block 165) and poured into a mold (block 175), and a stick placed into the mold (block 185). The molded mixture is allowed to cool and solidify around the stick (block 195), forming the lollipop. Alternatively, the step of placing the stick into the mold (block 185) may be deleted, and the content of the mold provided as a lozenge.

The order in which the various components are added and mixed may be varied, but it is preferable that methlycobalamin be added as the last component to minimize the amount of heat it is subjected to. In one embodiment, the mixture may be heated to about 100° C. or less, which is generally sufficient to melt the candy base, and does not degrade the methylcobalamin.

To administer methylcobalamin in adults, in one embodiment about 3.6 mg of methylcobalamin together with about 100 μg of folinic acid per lollipop may be administered orally once per day. Either or both methylcobalamin and folinic acid may be diluted or concentrated to achieve a desired dosage. If pharmaceutically acceptable salts of methylcobalamin and/or folinic acid are used, the amount of these salts is equivalent to the molar amount of methylcobalamin and folinic acid indicated.

In other embodiments, methylcobalamin can be administered from about 500-1500 μg per lollipop, preferably from about 1000-1250 μg per lollipop. In addition, folinic acid can be administered from about 25-300 μg per lollipop, preferably from about 25-150 μg per lollipop; more preferably from about 25-125 μg per pop, and even more preferably from about 25-100 μg per pop. In certain embodiments, methylcobalamin is administered without folinic acid.

In some embodiments for children, about half a lollipop can be administered once per day. If pharmaceutically acceptable salts of methylcobalamin and/or folinic acid are used, the amount of these salts is equivalent to the molar amount of methylcobalamin and folinic acid indicated. Again, in other embodiments, methylcobalamin is administered without folinic acid.

Methylcobalamin is preferably administered after a meal, more preferably after breakfast (since it can provide a feeling of energy throughout the day).

A person can conduct initial self-trials later in the afternoon when he or she begins to feel tired, which can provide a better idea of the difference pre- and post-administration. It is well known that a deficiency of vitamin B12, of which methylcobalamin is one form, may contribute to a range of symptoms such as fatigue, depression, and poor memory. Energy and mood elevation may then result from administration of methylcobalamin.

In other embodiments, the dose can be 3,600 μg methylcobalamin and 300 μg of folinic per lollipop (or lozenge). For people sensitive to folinic acid, a lower dosage of folinic acid may be initially used, or methylcobalamin can be used by itself, before optionally increasing the folinic acid dosage.

Due to biochemical, physiological and environmental variations, each person's need for methylcobalamin is different. As little as necessary to obtain the desired results is preferred since using more does not necessarily mean better results. Halting administration from time to time to monitor the efficacy or trying to space out administration may be good practice in determining an appropriate dosage. In addition, greater or lesser dosages than the dosages described above can be appropriate depending on the person.

A regimen for lollipop administration of methylcobalamin-containing lollipops includes placing the lollipop in the user's mouth. The user may suck on the lollipop or lozenge as one would a normal candy. For greater absorption of the dosage, it is advised that the user should not bite or chew, but rather rely on absorption by gradual dissolution in the mouth over several minutes.

In other embodiments, the lollipop can contain other vitamins, minerals and amino acids, such as, for example, zinc, omega 3 fatty acids (for example, from cod liver oil), other B vitamins such as B6 and pyridoxal 5-phosphate (P5P), B2, and B5, calcium, magnesium, vitamins A, D, and K, taurine or selenium, or any combination thereof.

The present disclosure may be better understood by referring to the accompanying examples, which are intended for illustration purposes only and should not in any sense be construed as limiting the scope of the disclosure as defined in the claims appended hereto.

### Example 1

Several types of brain pathology can give rise to abnormally strong or persistent cortical theta waves compared to beta waves, when detected using electroencephalography (EEG). Qualitative EEG (QEEG) is a brain mapping procedure that records electrical activity within the brain. Theta waves are relatively slow brain waves occurring about 4-7 times per second (3.5-7.5 Hz). Beta waves are electrical waves in the frequency range of 13-21 Hz (and as high as about 30 Hz).

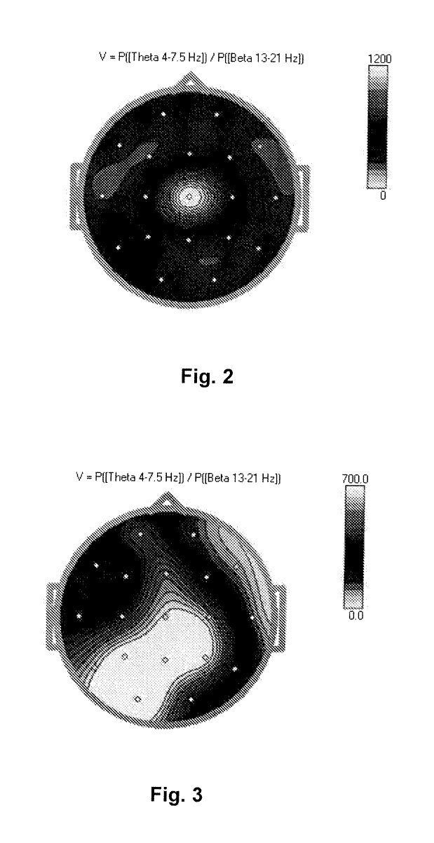

A quantitative electroencephalography (QEEG) and standardized low resolution electromagnetic tomography (LRET) evaluations were performed on an 8 year old male with an ASD diagnosis. The QEEG and LRET evaluations were performed prior to administration of a lollipop containing a dosage of 3.6 mg methylcobalamin, and again 45 minutes after administration. FIG. 2 shows the QEEG theta wave/beta wave activity ratio before lollipop administration. FIG. 3 show QEEG theta wave/beta wave activity ratio after lollipop administration.

Referring to FIG. 2, the initial evaluation (pre-administration) indicated atypical frequency maxima distribution with a presence of delta in the left and right parietal sites. The theta/beta peak power ratio in the eyes closed condition was found to be 12.0. Normative database comparison analysis indicates elevations of delta and theta across the anterior and central sites and elevations of alpha in the parietal and right temporal site. There were hyper coherence findings across all electrode pairs. Hyper coherence is an indicator of reduced well being. These patterns are consistent between the eyes closed and eyes open conditions however in the eyes open condition the theta/beta power ratio does decrease as does the alpha elevations indicating alpha reactants to eye opening.

Referring to FIG. 3, following lollipop administration, the QEEG spectral analysis indicated mildly atypical distribution of delta with a secondary focus at electrode site P4. The theta/beta peak power ratio at site CZ in the eyes closed condition dropped to 7.0. Normative database comparison analysis indicated mild elevations of delta in the parietal site, mild elevations of theta and alpha in the occipital and right parietal sites. These findings are consistent across both the eyes open and eyes closed condition, however in the eyes open condition normative database comparison analysis illustrates elevations predominantly of elevated delta diffusely across the cortex, elevations of anterior theta and occipital alpha.

In summary, following administration, the patient's atypical frequency maxima were reclassified mildly atypical, the theta/beta ratio dropped from 12.0 to 7.0, the elevations of delta and alpha waves were less severe, and all instances of hyper coherence across all electrode pairs were eliminated, indicative of elevated well being relative to pre-administration. Note the different scales in FIG. 2 and FIG. 3 for the intensity bar charts. The tests showed a dramatic reduction overall of theta wave activity after lollipop administration, while activity in the entire brain increased. The subject was observed to be more relaxed and attentive, while ocular “twittering” was greatly reduced.

### Example 2

An adolescent boy received methylcobalamin injections every day with little responsiveness. Two weeks after switching to one 3600 mcg methylcobalamin lollipop per day, the boy showed increased interest, such asking questions about the meaning of words like cooperate, possibility, responsible, etc., and demonstrated improved ability to put large concepts together, demonstrating reasoning and logic. His stamina improved, including engaging in outdoor activities all day.

### Example 3

An adult took one 3600 mcg dose lollipop a day for 2 months. Results included a: calming feeling, harder and stronger fingernails that used to be flaky and short, elimination of night vision glare, reduction in the size of a subcutaneous cyst on the subject's leg, and reduction of cellulite on the back of the legs.

### Example 4

3600 mcg dosage methylcobalamin lollipops were administered once per day by parents to a 5 yr old boy who had not previously had injections. Improvements noted included calmer disposition, improved evenness of temper, more relaxed appearance and improved ability to focus. One of the parents took one 3600 mcg dosage methylcobalamin lollipop in the morning, and noticed that at the end of the work day, she was no longer stressed and tired, as usual, with increased energy, a sense of well-being, without the crash that comes from caffeine. The parent attested to a sense of having a productive, pleasant day.

### Example 5

After taking injections, the parents of a young boy noticed a failure of continued response to the medication. The boy began a regimen of 3600 mcg dosage methylcobalamin lollipops and showed an increase in clarity and ability to verbalize immediately. The boy was more amenable to lollipops than the injections. The parents experimented by halting lollipop treatment for a week, and the boy again demonstrated trouble verbalizing and could not process what his parents were saying to him. After one day back on the lollipops the boy began using complete sentences, at which he was previously deficient, even while playing a video game.

### Example 6

A young girl in the habit of demonstrating defiant and uncooperative behavior was “bribed” with a methylcobalmin lollipop. Almost immediately the girl behaved much better and began to smile.

### Example 7

A 24 year old male on a gluten free/casein free (GF/CF) diet with PDD-NOS (Pervasive Developmental Disorder-Not Otherwise Specified)—one of the autism spectrum disorders) since the age of 5 began receiving a methylcobalamin lollipop. Observers noticed an immediate improvement in calmness, reduced obsessive and substantial reduction in the usual eye “twitter.” Since the subject was on a (GF/CF) diet, his acne cleared in 4 days, and he is a lot less stressful to be around.

### Example 8

An hour before presenting paper at a biomedical conference, an adult self-administered a methylcobalamin lollipop. The subject felt improved clarity, focus, and oral delivery, and reduced anxiety—challenges she typically faced before public speaking. The subject described the effect as “caffeine energy” without the “caffeine anxiety.”

### Example 9

A 40 year old women diagnosed with MS and suffering for decades with chronic neck/back pain, digestive issues, brain fog, fatigue, numbness asthma, etc., self-administered a methylcobalamin lollipop. Within minutes, the subject experienced increased energy, clarity, and mood elevation as a sense of “complete peace and joy.” The subject could breathe deeper than had been her experience for years and attested to an absence of pain.

### Example 10

A boy of 8 with autism spectrum disorder, who had not previously taken methlycobalmin injections, began a regimen of methylcobalamin lollipops. Within the first three weeks of use, the boy called his mother “Mom” for the first time, began attempting new words, and saying words daily. The boy appeared more focused, and sat in his classroom longer with fewer sensory integration breaks. His artwork has changed, making drawings (including a “self-portrait” and a Christmas tree) instead of scribbling or only writing the alphabet and numbers. The boy also demonstrates better memory recall, listening attention, following instructions, and improved interested in playing with games and interactive toys with others, including the parents. The boy now demonstrates a sense of humor, laughing appropriately at TV, and a more affectionate behavior, such as hugs, back rub/pats, and “playing with his parents' hair.”

### Example 11

After starting a regimen of methylcobalamin lollipops, a young male child began repeating every word his parents said to him (e.g., Spiderman, Daddy, Diaper). In one day of usage, the child's ability to echo words improved from about 5% to 90-100%.

When compared to alternative methods of administration, the methylcobalamin lollipop may have several advantages. The discomfort of injection is avoided. Nasal application is also less pleasant, and may not provide a reliably repeatable dose, because spray application can vary, where an uncontrolled portion of the dose may pass directly out of the nasal passage. Lollipop administration also provides unique access to the trigeminal nerve as result of constant and direct contact with the lingual nerve, a branch of the trigeminal ganglion.

What is claimed is:

1. A method of treating a psychological or neuro-physiological disorder, comprising administering orally a lollipop including methylcobalamin, or a pharmaceutically acceptable salt thereof, to a person in an amount sufficient to treat the disorder in the person.

2. The method of claim 1, wherein the disorder is selected from the group consisting of:
attention deficit hyperactivity disorder (ADHD), anxiety, depression, stress and chronic stress, socialization problems, mood problems, behavior problems, memory problems, dyslexia, depth perception problems, color viewing problems, visual and auditory processing problems, light modulation problems, night vision problems, speech problems such as finding words, apraxia, and articulation problems, sleep regulation problems, eye or muscle movement problems, chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, and autoimmune problems.

3. The method of claim 1, wherein the administered lollipop includes methylcobalamin in a dosage of about 0.1 mg to 10 mg.

4. The method of claim 1, wherein the administered lollipop further includes folinic acid or a pharmaceutically acceptable salt of folinic acid.

5. The method of claim 4, wherein the administered lollipop includes folinic acid in a dosage of about 25 mcg to 300 mcg.

6. The method of claim 1, further comprising administering one to three lollipops per day.

7. A lollipop for orally treating a psychological or neurophysiological disorder, comprising methylcobalamin, or a pharmaceutically acceptable salt thereof, in an amount sufficient to treat the disorder in the person.

8. The lollipop of claim 7, wherein the disorder is selected from the group consisting of: attention deficit hyperactivity disorder (ADHD), anxiety, depression, stress and chronic stress, socialization problems, mood problems, behavior problems, memory problems, dyslexia, depth perception problems, color viewing problems, visual and auditory processing problems, light modulation problems, night vision problems, speech problems such as finding words, apraxia, and articulation problems, sleep regulation problems, eye or muscle movement problems, chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, and autoimmune problems.

9. The lollipop of claim 7, further comprising methylcobalamin in a dosage of about 0.1 mg to 10 mg.

10. The lollipop of claim 7, further comprising folinic acid or a pharmaceutically acceptable salt of folinic acid.

11. The lollipop of claim 10, further comprising folinic acid in a dosage of about 25 mcg to about 300 mcg.

12. A method of making a lollipop for orally treating a psychological or neuro-physiological disorder comprises:
providing a candy base;
heating the candy base and to form a liquid;
adding to the liquid in a selected amount at least one of methylcobalamin and a pharmaceutically acceptable salt thereof;
mixing the liquid containing the methylcobalamin additive;
pouring the liquid and methylcobalamin mixture into a mold;
inserting a stick to the mixture in the mold to form the lollipop; and
allowing the mixture to cool and solidify around the stick.

13. The method of claim 12, wherein the selected amount of methylcobalamin additive results in a dosage of about 0.1 mg to 10 mg per lollipop.

14. The method of claim 12, further comprising adding to the candy base and flavoring before heating a selected amount of at least one of folinic acid and a pharmaceutically acceptable salt of folinic acid.

15. The method of claim 14, wherein the selected amount of folinic acid and/or salt thereof results in a dosage of about 25 mcg to about 300 mcg per lollipop.

16. The method of claim 12, further comprising adding at least one of omega 3 oil, fatty acids, B vitamins such as B6 and pyridoxal 5-phosphate (P5P), B2, and B5, vitamins A, D, and K, minerals such as zinc, calcium, magnesium, taurine and selenium.

17. The method of claim 12, comprising heating the candy base and flavoring to between the melting point of the candy base and not more than 100° C.

18. The method of claim 12, comprising adding a flavoring and/or a dye coloring to the candy base before adding the methylcobalamin and/or pharmaceutical salt thereof.

19. The method of claim 12, comprising adding at least one or more adjuvant, excipient and auxiliary substance before pouring the liquid and methylcobalamin mixture into the mold.

20. The method of claim 12, wherein no stick is placed in the mold and the mold content is provided as a lozenge.
